(19) United States
(12) Patent Application Publication
(10) Pub. No.: US 2006/0228369 A1
(43) Pub. Date: Oct. 12, 2006

(54) Stabilization and preservation of temperature-sensitive vaccines

(75) Inventor: Dexiang Chen, Seattle, WA (US)

Correspondence Address: STERNE, KESSLER, GOLDSTEIN & FOX PLLC, 1100 NEW YORK AVENUE, N.W., WASHINGTON, DC, 20005, US

(73) Assignee: Program for Appropriate Technology in Health

(21) Appl. No.: 11/266,496

(22) Filed: Nov. 4, 2005

Related U.S. Application Data
(60) Provisional application No. 60/669,955, filed on Apr. 11, 2005.

(51) Int. Cl. A61K 39/00

(52) U.S. Cl. 424/184.1

(57) ABSTRACT

The present invention relates to the use of temperature protective agents to protect temperature sensitive vaccines. Such agents can be used to protect vaccines from degradation that results from exposure to freezing conditions or hot environments. Such agents can also be used to help reduce or prevent degradation or contamination in reconstituted vaccines. The temperature protective agent may include glycerin, polyethylene glycol or propylene glycol.

### BACKGROUND OF THE INVENTION

1. Field of the Invention

The present invention relates generally to the use of temperature protective agents to protect temperature sensitive vaccines. Such agents can be used to protect vaccines from freezing or freeze damage as well as degradation in hot environments. Such agents can also be used to help reduce or prevent contamination in multidose vials of vaccine.

2. Background Art

Many current human and animal vaccine products contain an aluminum salt or its equivalent, such as aluminum hydroxide, aluminum phosphate, aluminum potassium sulfate or calcium phosphate. The function of the aluminum salt is to boost the immunogenicity and the efficacy of the vaccine antigens. Such aluminum adjuvant-containing vaccines are typically liquid formulations and are preferably stored between about 2° C. to about 8° C. Although such vaccines may be stable for a number of days, or possibly weeks at ambient temperature, such vaccines are typically kept between about 2° C. to about 8° C. to increase the time for which they can be stored without degradation.

Temperature sensitive vaccines, such as aluminum adjuvant-containing vaccines, are extremely sensitive to freezing. Freezing causes irreversible damage to the physical structure of the aluminum salt and loss of its adjuvant effect. As a result, a frozen vaccine may lose part or all of its potency. Indeed, vaccine labels and World Health Organization guidelines state that vaccines having an aluminum adjuvant component must not be frozen and any such vaccine should be discarded if thought to have been frozen.

Inadvertent freezing of vaccines in developing countries and even in developed countries is quite common due to lack of well-functioning cold chain equipment or improper handling in the transportation and storage of vaccines. Such freezing may be undetectable, and inadvertent freezing of vaccines often leads to the unknowing administration of damaged vaccines into humans, thereby mitigating the protection of the vaccines. Preventing the freeze-damage of vaccines is considered a global public health priority, and vaccines that are able to withstand temperatures below the recommended range of 2-8° C. are desired.

Temperature sensitive vaccines, such as aluminum adjuvanted-vaccines, are also susceptible to degradation at elevated temperatures, such as temperatures up to 55° C. Such elevated temperatures are likely to occur in hot, arid regions including northern Africa, equatorial locations, or during transportation where cold temperatures cannot be maintained.

Prior methods for avoiding temperature associated degradation of vaccines are inadequate. One approach is lyophilization, the conversion of liquid formulations into dry powders using a dehydration process. Unfortunately, this widely used freeze-drying method is not suitable for vaccines containing aluminum salt adjuvants because the freezing temperature involved will damage the adjuvant.

Lyophilization has also presented problems for vaccines that do not contain an aluminum salt adjuvant. For example, live-attenuated vaccines (and some non-live vaccines), which do not contain an aluminum adjuvant, are often lyophilized because of their intrinsic instability. The lyophilized products are reconstituted with diluent immediately before administration. Because lyophilization is a time-consuming and capacity-limiting step of vaccine production, lyophilized vaccines are usually presented in multi-dose vials. Some global guidelines require that unused vaccines in a multi-dose vial be discarded within six hours of reconstitution due to the concerns of potential contamination and potency loss. This results in vaccine wastage, which can account for losses of 50% or more of the vaccine doses distributed.

Another inadequate prior method for avoiding temperature associated vaccine degradation involves spray-freeze-drying. This process involves snap freezing of microdroplets at extremely low temperature and has demonstrated some success in producing dry powders of aluminum salt adjuvanted vaccines. Alternatively, a dehydration method including spray-drying that does not involve freezing temperatures may be used to prepare stable dry powders from aluminum adjuvanted vaccines. However, these approaches have significant drawbacks. In particular, the dehydration technology for stabilizing aluminum adjuvant is not fully developed. Additionally, the cost of manufacturing dry powder products is much higher than liquid vaccines. Furthermore, changing from liquid to dry powder formulation requires extensive formulation development, clinical testing, and relicensing of existing vaccines. Finally, for standard administration by needle and syringe, dry formulations would require reconstitution, a step that should be avoided as much as possible because of wastage, added cost, complexity for users, and safety risk.

In the public health arena, a common approach for freeze-prevention is to strengthen and optimize the cold chain. Drawbacks of this approach include the expense associated with extending, updating and improving the cold chain equipment, monitoring the equipment, and training those using the equipment. Moreover, although cold chain improvement can minimize freeze-damage, such improvement will not eliminate the occurrence of freeze-damage. Additionally, cold chain improvements will not mitigate the freezing that occurs outside of the cold chain in colder climates.

There remains a need for compositions and methods for stabilizing temperature-sensitive vaccines, and specifically for compositions and methods for stabilizing aluminum or calcium adjuvanted vaccines.

### BRIEF SUMMARY OF THE INVENTION

The present invention is directed to a method of preventing damage to an adjuvant in a temperature sensitive adjuvanted liquid vaccine composition comprising adding a temperature protective agent to a first liquid vaccine composition to form a second liquid vaccine composition having a freezing point below the freezing point of the first liquid vaccine composition; wherein the first liquid vaccine composition comprises an antigen and an adjuvant. The addition of the temperature protective agent can lower the freezing point of the first liquid vaccine composition below about 0° C., or from about 0° C. to about −55° C. After the addition of the temperature protective agent, the temperature protective agent can comprise about 1% to about 80% by volume of the second liquid vaccine composition. The temperature protective agent can comprise glycerin, propylene glycol, or polyethylene glycol. Where the temperature protective agent is polyethylene glycol, the polyethylene glycol can have an average molecular weight ranging from 200 to 20,000 kD. In a preferred embodiment, the polyethylene glycol can have an average molecular weight of about 300. The adjuvant can be an aluminum salt such as aluminum hydroxide, aluminum phosphate or aluminum potassium sulfate. Alternatively, the adjuvant can be calcium phosphate. The first liquid vaccine composition can be a human or animal vaccine composition. An embodiment of this invention also comprises storing the second liquid vaccine composition at about 0° C. to about −55° C. to protect the second liquid vaccine composition from microbial growth.

In another embodiment, the present invention is directed to a method of stabilizing a temperature sensitive adjuvanted liquid vaccine composition when frozen comprising adding a temperature protective agent to a first liquid vaccine composition to form a second liquid vaccine composition, prior to freezing the first liquid vaccine composition; wherein the first liquid vaccine composition comprises an antigen and an adjuvant; and wherein the temperature protective agent comprises glycerin, propylene glycol or polyethylene glycol. Where the temperature protective agent is polyethylene glycol, the polyethylene glycol can have an average molecular weight from about 200 to 20,000 kD. In a preferred embodiment, the polyethylene glycol can have an average molecular weight of about 300 kD. The adjuvant can be stabilized against agglomeration or sedimentation after freezing and thawing the second liquid vaccine composition.

The present invention is also directed to a method of stabilizing a temperature sensitive adjuvanted liquid vaccine composition at high temperatures comprising adding a temperature protective agent to a first liquid vaccine composition to form a second liquid vaccine composition prior to exposing the first liquid vaccine composition to high temperatures; wherein the first liquid vaccine composition comprises an antigen and an adjuvant. The temperature protective agent can protect the second liquid vaccine composition from temperature damage at temperatures up to about 55° C., or from about 4° C. to about 55° C. The temperature protective agent can also inhibit microbial growth in the second liquid vaccine composition at temperatures up to about 55° C., or from about 4° C. to about 55° C. The temperature protective agent can be propylene glycol.

### BRIEF DESCRIPTION OF THE DRAWINGS/FIGURES

FIG. 1 depicts the effect of freeze-protection excipients on the fluorescence emission peak of hepatitis B vaccines after three freeze-thaw treatments.

FIG. 2 depicts the effect of propylene glycol on the fluorescence emission peak of hepatitis B vaccines after three freeze-thaw treatments and incubation at 45° C. for 21 days.

FIG. 3 depicts the effect of freeze-protection excipients on the immunogenicity of hepatitis B vaccine in mice. The vaccine contained either glycerin, PEG-300, propylene glycol or no freeze-protection excipient and was either not subjected to any freeze thaw cycles (closed bars) or subjected to three freeze-thaw cycles (open bars).

FIG. 4 depicts the effect of freeze-protection excipients on the stability of hepatitis B vaccine containing 50% excipients or saline control subjected to three freeze-thaw cycles or kept at 4° C. as control.

FIG. 5 depicts the effect of propylene glycol on the potency of Hepatitis B vaccine subjected to three freeze-thaw treatments followed by incubation at 45° C. for 21 days.

### DETAILED DESCRIPTION OF THE PREFERRED EMBODIMENTS

This invention describes the use of low freezing point chemicals, through their anti-freezing properties, to stabilize adjuvanted vaccines. The anti-freezing properties of these chemicals have not been used to stabilize vaccines containing aluminum adjuvants or their equivalents.

Low freezing point chemicals have been used to preserve heat-sensitive biological materials in research reagents. For example, reagents such as antibodies and enzymes are often preserved in solution containing a low-freeze-point chemical and stored at temperatures less than 0° C. without freezing. In such applications, the low freezing point chemicals function by retarding or preventing damage to the biological reagents arising from chemical reactions such as denaturation, degradation or oxidation that may occur if such reagents are stored at higher temperatures. Low freezing point chemicals have also been used to preserve mammalian cells, viruses, and bacteria in deep freezing conditions, by mitigating physical injury to such biological materials caused by ice crystals. However, low freezing point chemicals have not been used to prevent damage to the adjuvant of a temperature sensitive adjuvanted vaccine.

The immediate effect of freezing on an aluminum adjuvanted vaccine (or vaccines adjuvanted with equivalents of aluminum adjuvant) is damage of the adjuvant manifested as agglomeration and accelerated sedimentation. Although the mechanism of such freeze-damage is not fully understood, it is clearly not mechanistically related to the damage which arises when biological reagents or materials are frozen. Therefore, the historical use of low-freeze point chemicals with biological reagents and materials has not suggested use of such chemicals for stabilizing vaccines comprising aluminum adjuvants and the like, particularly when the stabilization effect is exerted on the adjuvant.

Temperature sensitive adjuvanted vaccine compositions can be damaged at temperatures below freezing point or from 4° C. to about 55° C. Damage includes degradation of vaccine or reductions in efficacy of the vaccine. Damage can be caused by sedimentation or agglomeration of the adjuvant or antigen. Damage can also be caused by denaturation or fragmentation of the antigen. Hence, a damaged vaccine is a vaccine which has lost some or its entire efficacy relative to its undamaged form. Preventing damage in a temperature sensitive vaccine includes stabilizing or protecting the vaccine at temperatures below freezing point and/or from 4° C. to about 55° C.

The present invention is directed to using temperature protective agents to protect human or animal vaccines against freeze damage by lowering the freezing point of the vaccine liquid composition or by protecting components within the vaccine liquid composition upon freezing. The present invention is also directed to using temperature protective agents to protect human or animal vaccines against damage caused by elevated temperatures (e.g., up to about 55° C.). The present invention is also directed to using temperature protective agents to protect vaccine compositions from microbial contamination. The present invention is also directed to the temperature protected vaccines resulting therefrom.

Temperature protective agents can be added as a component of a vaccine liquid composition at the time of manufacture of the vaccine composition. Alternatively, temperature protective agents can be a component of a diluent used to reconstitute lyophilized, dried or powdered forms of vaccine. The invention is a practical alternative over existing technologies for stabilizing adjuvanted vaccines (e.g., aluminum or calcium salt adjuvanted vaccines) as well as other freeze-sensitive vaccines and addresses an enormous public health challenge.

In general, the temperature protective agent can stabilize the adjuvant by depressing the freezing point of the liquid vaccine composition below about 0° C. In particular, the temperature protective agent can lower the freezing point of the temperature sensitive adjuvanted vaccine composition to about 0° C. to about −55° C. The temperature protective agent can also stabilize the chemical or physical properties of the adjuvant when the liquid vaccine composition is frozen. For example, the adjuvant can be stabilized against agglomeration or sedimentation upon thawing of the frozen liquid vaccine composition. Because the liquid vaccine composition can be stored at about 0° C. to about −55° C., the present invention also provides further protection of liquid vaccine compositions from microbial growth.

The present invention is also directed to methods of preventing temperature damage to a liquid vaccine during storage, transportation or use by using a temperature protective agent. The present invention is also directed to methods of extending the storage time of a dry or concentrated vaccine by adding a temperature protective agent as part of the diluent used to reconstitute a lyophilized, powder or concentrated form of a vaccine.

As used herein, “formulation” and “composition” are used interchangeably. Also as used herein, “temperature protective agents” refer to pharmaceutically acceptable excipients that can be added to a dry or liquid vaccine composition. The temperature protective agent can be a cold-protective agent or a heat-protective agent. “Cold-protective agents” refer to excipients that protect liquid vaccine formulations at cold temperatures by either i) preventing the liquid vaccine formulation from freezing by depressing its freezing point, or ii) protecting the liquid vaccine formulation's constituent vaccine or adjuvant when the liquid vaccine formulation is frozen. As used herein, the term “cold-protective agents” is interchangeable with the term “freeze-protection excipients” or “freeze-protective agents.” “Heat protective agents” refer to excipients that protect a liquid vaccine formulation's constituent vaccine or adjuvant at elevated temperatures. Cold-protective agents and heat-protective agents can be used in combination or separately in the liquid vaccine formulation. A temperature protective agent can serve as both a cold-protective agent and a heat-protective agent.

Temperature protective agents preferably have the following properties:
the temperature protective agent should be safe for parenteral administration in humans;
the temperature protective agent should be readily miscible or soluble with water; and
the temperature protective agent should not by itself cause any damage to the aluminum adjuvant, vaccine antigen, or other formulation components.

In one embodiment of the invention, the temperature protective agents do not support bacterial growth and are bacteristatic, bactericidal or antimicrobial.

In addition, cold protective agents preferably have the following properties:
the cold protective agent can have a freezing point that is significantly lower than that of water; and
the cold protective agent should remain miscible or admixed with water at cold temperatures or when frozen.

In addition, heat protective agents preferably have the following properties:
the heat protective agent is not so volatile at elevated temperatures so as to entirely escape the vaccine solution at standard atmospheric pressure; and
the heat protective agent should remain miscible or admixed with water at elevated temperatures.

Non-limiting examples of temperature protective agents that meet these criteria include pharmaceutically acceptable alcohols, polyols, amino acids and saccharides. Non-limiting examples of alcohols that can be used as temperature protective agents include ethanol, propanol, isopropanol, butanol, isobutanol, t-butanol, 2-methyl-1-propanol and other alcohols of C₄-C₈-alkyls. Polyols include diols, triols and chemicals having more than three alcoholic groups. Alcohols and polyols can also have carbonyl carbons (e.g., carboxylic acids, keto- or aldehyde-groups). Alternatively, alcohols and polyols can be compounds without any carbonyl carbons. Non-limiting examples of polyols include mannitol, sorbitol, erythritol, xylitol, maltitol, siomalt, lactitol. An amino acid temperature protective agent can be any pharmaceutically acceptable natural or derivatized amino acid, or an ester or salt thereof. Non-limiting examples of amino acids include glycine, glutamine, glutamic acid, aspartic acid, sodium glutamate, methionine, alanine, proline, arginine, tryptophan, lysine, and histidine. Non-limiting examples of saccharides include trehalose, sucrose, lactose, and raffinose. The temperature protective agent can also be sodium lactate. Particular examples of temperature protective agents include glycerin, propylene glycol and polyethylene glycol of various molecular weights. The skilled artisan is aware of other temperature protective agents that include the properties described above that can be used according to the present invention.

In one embodiment of the invention, the temperature protective agent can be glycerin. The freezing point of glycerin is less than −60° C. It can be readily mixed with water at any proportion. The freezing points of water-glycerin mixtures range from 0 (pure water) to −34° C. (60% glycerin-40% water). Glycerin is used in many medical products, including those injected intramuscularly. Glycerin has been used to preserve cells, organs for transplantation, and food under freezing conditions.

In an alternate embodiment of the invention, the temperature protective agent can be propylene glycol. The freezing point of propylene glycol is −60° C. It can be readily mixed with water in any proportion. The freezing points of water-propylene glycol mixture ranges from 0 (pure water) to −48° C. (60% propylene glycol-40% water). Propylene glycol is anti-bacterial and used in many medical products including those injected intramuscularly. Other applications of propylene glycol in the literature include preservation of cells, anti-icing fluid, and antifreeze fluid.

In an alternate embodiment of the invention, the temperature protective agent can be polyethylene glycol (PEG). The molecular weight of PEG ranges from 200 to 20,000 kD. As an example, PEG-300 has a very low freezing point and is bacteriostatic. PEG is used as freeze stabilizer in preserving cells, bacteria, seeds, and biomolecules.

The temperature protective agent is added to the vaccine formulation in a concentration sufficient to protect the vaccine antigen or the adjuvant from temperature damage. The concentration of the temperature protective agent can constitute 1% to 80% of the formulation. Preferably, the temperature protective agent comprises 1%, 5%, 10%, 15%, 20%, 25%, 30%, 35%, 40%, 45%, 50%, 55%, 60%, 65%, 70%, 75% or 80% of the formulation, or falls within a range defined by any two of these percentage values.

The temperature protective agent can be a cold protective agent that is preferably added in an amount to depress the freezing point of the liquid vaccine formulation to −1° C. to −75° C. In a preferred embodiment of the invention, the cold protective agent depresses the freezing point of the liquid vaccine formulation to less than 0° C., −5° C., −10° C., −15° C., −20° C., −25° C., −30° C., −35° C., −40° C., −45° C., −50° C. or −55° C.

Human vaccines that contain an aluminum salt adjuvant and are sensitive to freeze-damage include, but are not limited to tetanus toxoid, diphtheria toxoid, pertussis vaccine, hepatitis B vaccine, hepatitis A vaccine, inactivated polio vaccine, liquid haemophilus influenza b conjugate vaccine, type C meningococcal conjugate vaccine, and pneumococcal conjugate vaccine conjugate. Many vaccines under development, including type A meningococcal vaccines, HIV vaccine, malaria vaccine, human papilloma virus vaccine, herpes simplex virus vaccine, anthrax vaccine and others may include an aluminum adjuvant component. Human vaccines that are not freeze-sensitive, but are potentially subject to microbial contamination once reconstituted include lyophilized haemophilus influenza b conjugate vaccine, measles, measles-mumps-rubella, yellow fever, vericella, Japanese Encephalitis virus, and rotavirus vaccines.

The mechanism for freeze damage of liquid vaccine formulations is not fully understood. Under normal storage conditions, aluminum adjuvants are stable colloidal suspensions. The colloidal property is important for the adjuvant activity and the vaccine potency. After a single freeze-thaw cycle (in which a liquid vaccine formulation is frozen and then thawed) the colloidal properties of aluminum salt are lost and the aluminum salt becomes coagulated. The coagulated salt is seen as large aggregated clumps under a light microscope. The large aggregates precipitate rapidly in the solution, and can sometimes also be visually detected.

Sedimentation assay, which measure the rate of precipitation of aluminum salt, is normally used by vaccine manufacturers to characterize the quality of the vaccine products. Freeze-damaged vaccines have a much quicker sedimentation rate than the colloidal aluminum adjuvant formulations when they are first manufactured or properly stored.

In one embodiment of the present invention, temperature protective agents can be used to significantly lower the freezing temperature and therefore preserve many freeze-sensitive vaccines that are used to prevent or treat infectious diseases, cancer, autoimmune diseases, or allergies. The most notable vaccines are those that have an aluminum hydroxide, aluminum phosphate, aluminum potassium sulfate or calcium phosphate adjuvant. Other freeze sensitive vaccines such as inactivated polio vaccine can also benefit from this invention.

Inclusion of freeze-protective agent in the diluent for multi-dose vials of lyophilized vaccines (such as measles, measles-mumps-rubella, and yellow fever) potentially allows the reconstituted vaccine to be stored at much lower temperatures without freezing, including from −10° C. to −50° C., thereby reducing the risk of contamination and helping to maintain the vaccine potency. Even where freezing occurs, the freeze-protective agent stabilizes or preserves the vaccine formulation. Regardless of whether the vaccine formulation is frozen or not at 0° C. or less, microbial contamination due to microbial growth can be mitigated or eliminated. Thus, storage time of the reconstituted vaccine can be extended and the vaccine wastage can be reduced.

Hence, in another embodiment, the invention is directed to a kit for preventing the degradation or contamination of a vaccine comprising i) a vaccine composition comprising an antigen and an adjuvant; and ii) a temperature protective agent. Preferably, the vaccine composition is a dry composition and packaged separately from the temperature protective agent. In a preferred embodiment, the temperature protective agent is a packaged, sterile aqueous solution of glycerin, polyethylene glycol or propylene glycol. In this embodiment of the invention, the temperature protective agent is a component in the reconstituting diluent added to a dry, lyophilized or powder vaccine form. Alternatively, the temperature protective agent can be added to a dry, lyophilized or powder vaccine that has already been reconstituted with a diluent.

While specific embodiments are discussed in the following Examples, it should be understood that this is done for illustrative purposes only. A person skilled in the relevant art will recognize that the following Examples are not limiting and other embodiments can be used without departing from the spirit and scope of the invention.

### EXAMPLES

Example #1

### Freeze-Prevention and Preservation of the Colloidal Suspension of Aluminum Salt Solution

Table 1 shows various concentrations of glycerin and propylene glycol, and the associated temperatures at or above which aluminum-adjuvanted vaccines would be expected not to freeze.

TABLE 1
The freezing points of solutions resulting from various concentrations of
freeze-protection excipients in water.

| % in water (v/v)* | Propylene glycol | Glycerin |
|---|---|---|
| 10 | −3° C. | −2.2° C. |
| 20 | −8° C. | −5.3° C. |
| 30 | −14° C. | −8.8° C. |
| 40 | −22° C. | −17.2° C. |
| 50 | −34° C. | −21.4° C. |
| 60 | −48° C. | −34° C. |

*volume to volume

The objective of this study was to determine the protective effect of freeze protection agents on aluminum adjuvants. Preservation of the colloid nature of the adjuvant and the size of aluminum adjuvant particles are important for the adjuvant effect and the potency of the vaccine. Three freeze protection agents including glycerin, polyethylene glycol-300, and propylene glycol were evaluated for their ability to preserve the physical structure of aluminum hydroxide and aluminum phosphate adjuvants subjected to three freeze (−20° C.) and thaw (24° C. room temperature) cycles.

Method: In a matrix setup, aluminum hydroxide (Accurate Chemicals & Scientific Corporation, Westbury, N.Y.) or aluminum phosphate (Accurate Chemicals) was combined with glycerin, polyethylene glycol-300, or propylene glycol (all purchased from Sigma Chemicals, St. Louis, Mo.) in 13×100 mm culture tubes. Each freeze protection agent was tested at 50% final concentrations in saline and the final adjuvant concentration was 0.2% (v/v). Saline was used as a diluent for both the freeze protection agents and the adjuvants. The test samples were subjected to three freeze (−20° C. for 4-20 hrs) and thaw (room temperature for 1-2 hrs) cycles. Controls of identical composition were set up in parallel and subjected to three cycles of exposure to 4° C. for 4-20 hours and room temperature for 1-2 hours. An additional control included aluminum adjuvants in saline without a freeze-protection agent. Three pieces of data were collected for each test and control sample: the freezing status after each exposure to −20° C. (or 4° C. for the control), the sedimentation rate after each thaw, and the agglomeration (i.e., aggregation of small particles into large clumps) of the aluminum salt particles after the third freeze-thaw event.

Examination of the samples: At the end of each freeze period, all tubes were removed from their respective environments and examined visually to determine whether freezing had occurred by inverting each tube and observing the material. After the visual examination, the tubes were allowed to thaw and equilibrate to room temperature for a minimum of one hour. The content of all tubes was mixed by gentle shaking, replacing the tubes in the test tube rack and observing over one hour at 15-minute intervals for precipitation as evidenced by a clear supernatant. At each time point, the supernatant was measured from the top of the liquid meniscus to the top of the precipitant. After the measurements were taken, the tubes were mixed by gentle shaking and returned to their respective environments. The last thaw cycle had an additional 120-minute time point added to assess the completion of precipitation.

At the end of the third freeze-thaw cycle, light microscopic examination of the aluminum structure was performed at 400× magnification, and compared to saline controls for agglomeration of particles. Agglomeration is an indication of aluminum adjuvant damage.

Results: Several important observations were made from this study. First, all three freeze protection agents at 50% concentration completely prevented freezing of aluminum hydroxide and aluminum phosphate solution when exposed to −20° C. for up to 20 hours in each of the three freeze-thaw cycles, as shown in Table 2. Control adjuvant in saline (without a freeze protection agent) were 100% frozen. Second, after three freeze-thaw cycles, all three freeze protection agents at 50% concentration completely prevented agglomeration of aluminum hydroxide and aluminum phosphate. As a control, freezing of aluminum adjuvant in saline without a freeze protection agent resulted in complete agglomeration of aluminum salt particles. Third, the sedimentation rate of aluminum salts after each freeze-thaw in the presence of 50% freeze protection agent was unchanged when compared to controls of the same composition kept at 4° C. In contrast, the saline control had much quicker sedimentation rate starting from the first freeze-thaw event. At the end of the three freeze-thaw cycles, the aluminum salts in saline controls had complete sedimentation at 15 minutes. The tubes containing a freeze-protection agent had no or partial sedimentation at the 2 hour time point as shown in Table 2. It should be noted that propylene glycol and polyethylene glycol may have a slight effect on the sedimentation rate of aluminum adjuvant solution kept at 4° C., which is neither worsened nor improved when exposed to −20° C. and does not appear to have any negative effect on the physical-chemical properties of the aluminum adjuvant. Freeze protection agents at appropriate concentrations can prevent freezing of aluminum salt adjuvants, and fully preserve their physical properties and structure that are important for their adjuvant effect.

TABLE 2
Preservation of the aluminum adjuvant's physical properties by freeze
protection excipients at 50% concentration.

| | | | % sedimen- | |
|---|---|---|---|---|
| | | | tation | |
| | FPA^{a} | % | at | |
| Adjuvant | (50%) | frozen^{b} | 120 min^{c} | Agglomeration^{d} |
| Alum hydroxide | Saline | 100 | 86 | yes |
| Alum hydroxide | Glycerin | 0 | 0 | no |
| Alum hydroxide | PEG-300 | 0 | 14 | no |
| Alum hydroxide | PPG | 0 | 75 | no |
| Alum phosphate | Saline | 100 | 82 | yes |
| Alum phosphate | Glycerin | 0 | 0 | no |
| Alum phosphate | PEG-300 | 0 | 10 | no |
| Alum phosphate | PPG | 0 | 24 | no |

^{a}FPA: freeze protect agent.
^{b}The % content of the formulation that was frozen, which is the same for all three freeze events.
^{c}The % sedimentation at 120 minutes after the third freeze-thaw event
^{d}The agglomeration after the third freeze-thaw event.

### Example #2

### Preservation of the Particle Size of Aluminum Adjuvant After Freeze-Thaw Treatments

Methods: This study used a quantitative assay to measure the particle size distribution of aluminum adjuvant before and after freezing-thaw treatments. A commercial human hepatitis B vaccine (Shantha Biotech, Hyderabad, India) was used. Each milliliter of vaccine contains 20 μg of yeast recombinant hepatitis B surface antigen adsorbed to 500 μg of aluminum hydroxide. Hepatitis B vaccine was formulated with saline, 50% glycerin, 50% PEG-300, or 50% propylene glycol and then subjected to three freeze-thaw treatments. Freezing took place in a −20° C. freezer for 18 hours and thawing took place on the laboratory bench at 24° C. for 4 hours. Hepatitis B vaccine with saline (no excipient) became frozen; all other formulations did not freeze. Particle sizing of samples following three freeze-thaw treatments was conducted using a Coulter Counter® Model Z1 (Beckman Coulter). Prior to beginning the study, the instrument was calibrated using the Standards Mixed Kit (Beckman Coulter) which contained standards for particles of 2 μm, 5 μm, 10 μm, 20 μm, and 43 μm. For each formulation and thermal treatment, measurements of particles from three separate vials were obtained. The samples were prepared as follows. First, the vaccine in the vial was gently resuspended. Next an aliquot of the vaccine (100 μl for the control and 200 μl for all other samples) was diluted into 20 ml of ISOTON® II diluent (Beckman Coulter). The diluted suspensions were then gently mixed until they appeared homogeneous. Next they were assayed for particles in the following size ranges: 1.5-3 μm, 3-6 μm, 6-9 μm, 9-15 μm, 15-20 μm, 20-25 μm, and 25-30 μm. Six readings per size range were obtained for each sample. The total particles per ml were obtained by summing the particles per ml in each size range. This total was then used to obtain the percentage of particles in each size range per sample. The average of the three separate samples per formulation and thermal treatment were tabulated.

Results: The particle sizing data indicate that the freeze-thaw treatment affected the size of the antigen/adjuvant complexes (Table 3). In the absence of freezing and thawing, 99.81% of the particles were in the 1.5-3 μm size range. Most of the remaining particles were in the 3-6 μm size range. There were only a few particles of size 6-9 μm. In the absence of excipients, freeze-thawed samples had only 75% of the particles in the sample of size 1.5-3 μm. Moreover, these samples had measurable particles in all size ranges assayed (Table 3) and fewer particles overall due to the formation of large aggregates (data not shown). We have seen that the agglomeration of aluminum adjuvant is associated with the loss of the immunogenicity of hepatitis B vaccine (Example #7 below). When this sample is analyzed by light scattering, the average particle size is 30 μm (data not shown), and this is significantly larger than the 3 μm size of the standard vaccine prior to freeze-thaw treatments. When freeze-protecting excipients were included in the formulations, the particle size distribution resembled the 4 ° C. control vaccine. This result indicated all three excipients at 50% effectively protect the particle size change of the aluminum adjuvant at −20° C. freezing temperatures.

TABLE 3
Effect of freeze-thaw treatments and freeze protection excipients on the
size of particles

| | Percentage of particles in each size range(μm) | | | | | | |
|---|---|---|---|---|---|---|---|
| Sample | 1.5-3 | 3-6 | 6-9 | 9-15 | 15-20 | 20-25 | 25-30 |
| Control, 4° C., | 99.81 | 0.18 | 0.01 | 0 | 0 | 0 | 0 |
| Control (no excipient), FT | 75.01 | 20.71 | 3.25 | 0.65 | 0.25 | 0.09 | 0.04 |
| 50% PEG-300, FT | 99.67 | 0.31 | 0.02 | 0 | 0 | 0 | 0 |
| 50% Propylene glycol, FT | 99.77 | 0.21 | 0.01 | 0 | 0 | 0 | 0 |
| 50% Glycerin, FT | 99.54 | 0.46 | 0.01 | 0 | 0 | 0 | 0 |

FT: freeze-thaw

### Example #3

### Preservation of the Particle Size of Aluminum Adjuvant After Freeze-Thaw Treatments and Heat Exposure

An accelerated stability study was conducted to determine if the particle sizes of aluminum adjuvant that have gone through freeze-thaw treatment would change during storage. Hepatitis B formulations containing saline, 50% glycerin, 50% PEG-300, or 50% propylene glycol were subjected to three freeze-thaw treatments or kept at 4° C. (as control) and then all formulations were incubated at 66° C. for 14 days. Freezing took place in a −20° C. freezer for 18 hours and thawing took place on the laboratory bench at 24° C. for 4 hours. Control hepatitis B with saline (no excipient) became frozen; all other formulations did not freeze. The size distributions of the particles indicate that freezing in the absence of excipients is most detrimental to maintaining the particle size distribution (Table 4). When excipients (e.g., PEG-300, propylene glycol, or glycerin) were present in the sample, the size distribution was similar to the control, regardless of the thermal treatment. This data indicated that all three temperature protection excipients preserve the particle size distribution of aluminum adjuvant when the vaccines are exposed to freezing temperature and upon storage at high temperatures.

TABLE 4
Effect of freeze-thaw treatment followed by storage at 66° C. for 14 days on
the size distribution of particles.

| | Percentage of particles in each size range(μm) | | | | | | |
|---|---|---|---|---|---|---|---|
| Sample | 1.5-3 | 3-6 | 6-9 | 9-15 | 15-20 | 20-25 | 25-30 |
| Control,: 4° C., | 99.74 | 0.24 | 0.02 | 0 | 0 | 0 | 0 |
| Control, FT, 66° C. | 79.43 | 16.19 | 3.08 | 0.90 | 0.26 | 0.08 | 0.05 |
| Control, 4° C., 66° C. | 99.71 | 0.25 | 0.03 | 0.01 | 0 | 0 | 0 |
| Vaccine + 50% PEG-300, FT, | 99.53 | 0.43 | 0.03 | 0.01 | 0 | 0 | 0 |
| 66° C. | | | | | | | |
| Vaccine + 50% PEG-300, 4° C., | 99.68 | 0.30 | 0.02 | 0.01 | 0 | 0 | 0 |
| 66° C. | | | | | | | |
| Vaccine + 50% propylene | 99.66 | 0.32 | 0.02 | 0.01 | 0 | 0 | 0 |
| glycol, FT, 66° C. | | | | | | | |
| Vaccine + 50% propylene | 99.73 | 0.25 | 0.01 | 0.01 | 0 | 0 | 0 |
| glycol, 4° C., 66° C. | | | | | | | |
| Vaccine + 50% glycerin, FT, | 99.74 | 0.23 | 0.02 | 0.01 | 0 | 0 | 0 |
| 66° C. | | | | | | | |
| Vaccine + 50% glycerin, 4° C., | 99.76 | 0.21 | 0.01 | 0.01 | 0 | 0 | 0 |
| 66° C. | | | | | | | |

FT: freeze-thaw

### Example #4

### Preservation of the Particle Size of Aluminum Adjuvant Despite Freezing of Aqueous Aluminum-Adjuvanted Hepatitis B Vaccine Formulations

Method: These studies were to determine if freeze-prevention is absolutely required to prevent freeze-damage of aluminum-adjuvanted vaccines. In the first study, hepatitis B vaccine containing 50%, 40%, 30%, 20%, 10%, 5%, and 0% glycerin was subjected to three freeze (−20° C.) and thaw (24° C.) cycles as previously described. The samples were then diluted 10× with water. Samples were assayed using the dynamic light scattering mode of a Nicomp 380 instrument in the Gaussian mode. In the second study, hepatitis B vaccine containing 50%, 30%, 10%, and 0% propylene glycol was subjected to three freeze (−20° C.) and thaw (24° C.) cycles. The size of the antigen and adjuvant complex was measured using a Coulter Counter®.

Results: The effects of glycerin concentration on the particle sizes of aluminum adjuvant are presented in Table 5. It was found that particle size of the aluminum adjuvant and antigen complex could be effectively maintained at all glycerin concentrations tested (5 to 50%). Glycerin concentrations as low as 10% maintained the particle size to that of vaccine in 50% glycerin. By using a glycerin concentration of 5%, the particle size increased slightly but was similar to the unfrozen control and was still well below that of the vaccine particles that were subjected to freezing and thawing without added glycerin. Vaccine containing less than 40% glycerin freezes at −20° C., but maintains the normal size of aluminum adjuvant. This data indicated that some excipients at low concentration may not prevent vaccine freezing, but are still effective in protecting the vaccines and aluminum adjuvant from freeze damage.

TABLE 5
Effect of glycerin concentration on size of the aluminum adjuvant particles
following 3 freeze-thaw cycles

| | Volume Weighting | |
|---|---|---|
| | Mean Diameter | |
| | (nm) | SD (nm) |
| Control, 4° C. | 3477 | 1078 |
| Vaccine + 50% glycerin, FT | 1692 | 393 |
| Vaccine + 40% glycerin, FT | 1908 | 394 |
| Vaccine + 30% glycerin, FT | 2162 | 532 |
| Vaccine + 20% glycerin, FT | 2397 | 727 |
| Vaccine + 10% glycerin, FT | 2509 | 641 |
| Vaccine + 5% glycerin, FT | 3331 | 1615 |
| Vaccine + 0% glycerin, FT | 25682 | 20062 |

FT freeze-thaw

The stabilization effect of propylene glycol on the aluminum particles is shown in Table 6. When exposed to −20° C., the control vaccine and vaccines with 10% and 30% propylene glycol became completely frozen. The vaccine with 50% propylene glycol did not freeze. However, all three concentrations of propylene glycol helped to maintain the size distribution of the aluminum adjuvant. Thus, freeze protection excipients at high concentration can stabilize aluminum adjuvanted vaccines by preventing freezing; and excipients at low concentrations, although ineffective in preventing freeze, can protect the aluminum adjuvant from the freeze-damages.

TABLE 6
Effect of propylene glycol concentration on size distribution of the
aluminum adjuvant particles following 3 freeze-thaw cycles

| | Average number of particles in each size range(μm) | | | | | | |
|---|---|---|---|---|---|---|---|
| Sample | 1.5-3 | 3-6 | 6-9 | 9-15 | 15-20 | 20-25 | 25-30 |
| Control vaccine, 4° C. | 99.74 | 0.24 | 0.01 | 0.00 | 0.00 | 0.00 | 0.00 |
| Control vaccine + 0% | 70.10 | 22.16 | 4.50 | 2.60 | 0.48 | 0.07 | 0.02 |
| propylene glycol, FT | | | | | | | |
| Vaccine + 50% | 99.57 | 0.38 | 0.03 | 0.01 | 0.00 | 0.00 | 0.00 |
| propylene glycol, FT | | | | | | | |
| Vaccine + 30% | 99.50 | 0.43 | 0.04 | 0.02 | 0.00 | 0.00 | 0.00 |
| propylene glycol,: FT | | | | | | | |
| Vaccine + 10% | 99.35 | 0.58 | 0.05 | 0.02 | 0.00 | 0.00 | 0.00 |
| propylene glycol, FT | | | | | | | |

### Example #5

### Preservation of the Structure of the Hepatitis B Surface Antigen After Freeze-Thaw Treatment

Methods: In addition to the aluminum adjuvants, another important component of vaccine formulation is the vaccine antigen. It is important that the freeze-protection excipients do not have any adverse effect on the structure and stability of the vaccine antigen. Here, this is studied using the human hepatitis B vaccine. Fluorescence Spectroscopy was used to determine the changes in the tertiary structure of the hepatitis B surface antigen in vaccine formulations that have undergone freeze-thaw treatments in the presence of freeze-protection excipients. Vaccines containing no excipient (saline), 50% glycerin, 50% PEG-300, or 50% propylene glycol were subjected to three freeze-thaw treatments as previously described. The control vaccine was kept at 4° C. The samples (approximately-1.8 ml) were transferred to triangular fluorescence cuvettes and allowed to settle overnight (minimum 16 hours) at 4° C. The next day, fluorescence spectra were obtained as follows. The cuvettes were positioned in the fluorometer such that the excitation beam hit the antigen/adjuvant layer that had formed in the bottom portion of the cuvette. Samples were excited with a wavelength of 280 nm and emission spectra were obtained to monitor the fluorescence of the antigen's tyrosine and tryptophan residues. All spectra were collected at 25° C. using a QM-4 fluorometer (Photon Technology International, Inc) with a Peltier temperature-controlled cuvette holder (Quantum Northwest). Both the emission and excitation slit widths were set at 3 nm. Data was collected at 1 nm increments with 1 second averaging time per nm. To assist in spectral comparisons, all spectra were normalized to the same maximum value by dividing all data in each spectrum by the highest intensity value. The peak position of samples from three separate vials per formulation and thermal treatment were averaged.

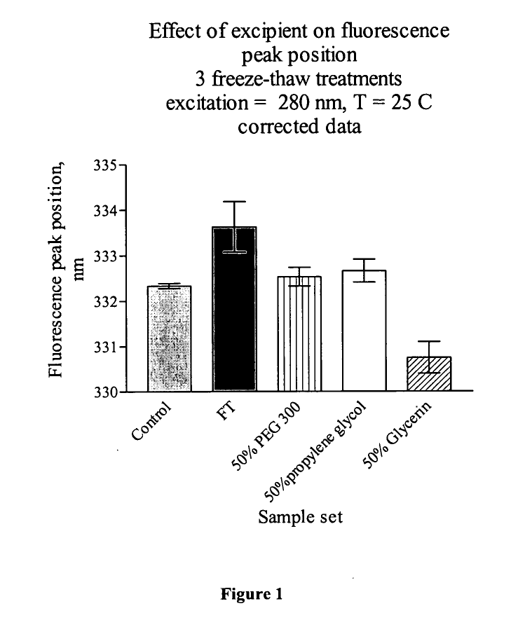

Results: FIG. 1 displays averaged normalized fluorescence emission peak of vaccine not subjected to freeze-thaw cycle (control) or following three-freeze-thaw cycles (FT). Hepatitis B vaccine containing no excipient, 50% propylene glycol, 50% PEG-300, or 50% glycerin were subjected to three freeze-thaw treatments. Data represent average (n=3) fluorescence emission at 280 nm using a QM-4 fluorometer at 25° C. The control is the standard vaccine stored at 4° C.

The polarities of the environment of the tryptophan and tyrosine residues are affected by the freeze-thaw (FT) treatment (control versus FT-no excipient). This changes was prevented by the addition of 50% PEG-300 or 50% propylene glycol (control versus FT-50% stabilizer). There was a slight blue shift of the fluorescence emission peak in the case of 50% glycerin, suggesting that the aromatic residues are in a somewhat less polar environment in the presence of 50% glycerin. This result indicates that all three freeze-protection excipients are compatible with the hepatitis B surface antigen and prevented the structural changes of the protein during freeze-thaw.

### Example #6

### Preservation of the Structure of the Hepatitis B Surface Antigen After Freeze-Thaw Treatment and Incubation at 45° C. for 21 Days

Method: Hepatitis B vaccine containing 50%, 30%, 10%, and 0% propylene glycol was subjected to three freeze (−20° C.) and thaw (24° C.) cycles and then incubated at 45° C. for 21 days. The fluorescence emission peaks (n=3) at 280 nm were collected using a QM-4 fluorometer to determine if the excipients have any adverse effect on the structure of hepatitis B surface antigen after storage at elevated temperature. The control is the standard vaccine stored at 4° C.

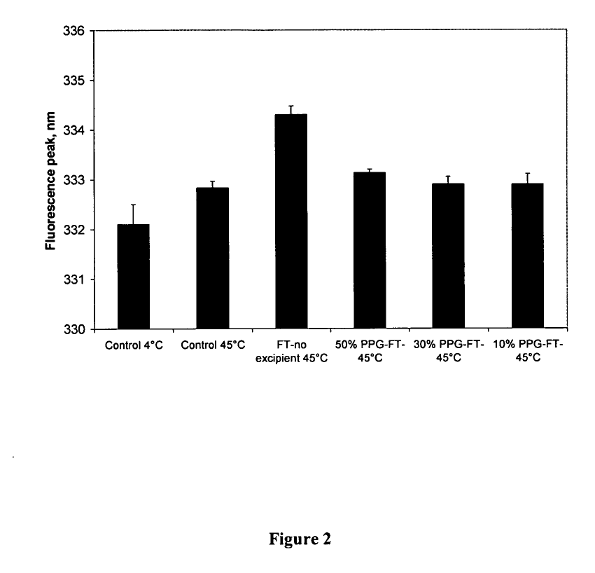

Results: In the absence of an excipient (FIG. 2), no significant shift of the fluorescence emission peak was noticed when the control vaccine (was incubated at 45° C. for 21 days (control 45° C.). The freeze thaw control (no excipient) had significant shift when incubated at 45° C. for 21 days, indicating thermal-induced structural changes of the vaccine. When the vaccine was formulated with 50%, 30%, or 10% PPG, the fluorescence peaks were not shifted when compared to the control stored at 4° C. or 45° C. These results indicate that propylene glycol effectively preserves the structure of hepatitis B surface antigen against both freeze-thaw treatment and at elevated temperature.

### Example #7

### Preservation of the Immunogenicity of Hepatitis B Vaccine After Freeze-Thaw

Method: Hepatitis B vaccine (Shantha Biotech, India) was combined with glycerin, propylene glycol, polyethylene glycol, or saline as a control at a 1:1 ratio to give a final excipient concentration of 50% (v/v). Each mixture was divided into two fractions; one fraction was kept at 4° C. and the other subjected to three freeze-thaw treatments. Freezing took place in a −20° C. freezer for 18 hours and thawing on the laboratory bench at 24° C. for 4 hours. Only the saline-diluted hepatitis B vaccine became solidly frozen. After the three freeze-thaw treatments, the vaccine was diluted 1 to 5 with saline (1 part vaccine and 4 part saline) and used to immunize mice.

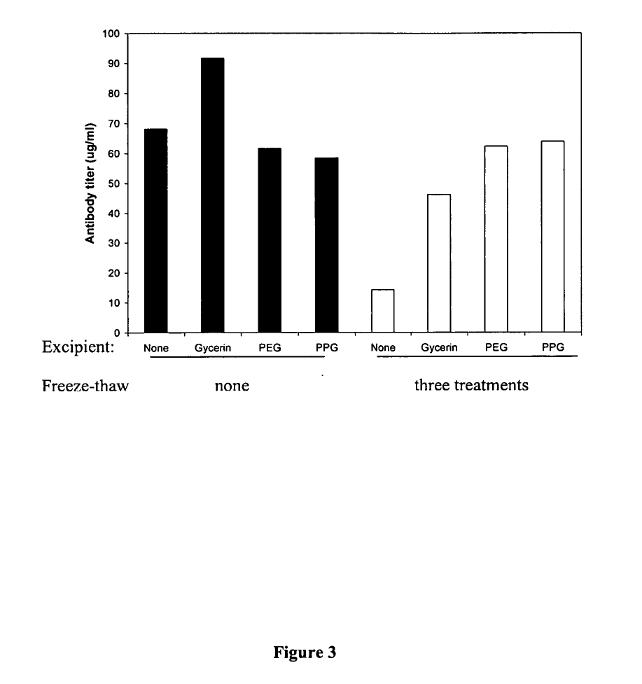

Six week old female Balb/C mice were used to study the immunogenicity of the above vaccines. Nine (9) groups of mice (8 animals per group) were used as depicted in FIG. 3. Each animal was injected intraperitoneally with 0.5 ml vaccine containing 1 μg of vaccine antigen and 0.03 mg of aluminum hydroxide using a 27-gauge needle on days 0 and 28. Blood samples were collected before each immunization and 14 days after the last immunization. Serum was collected after centrifugations of the clotted blood and kept frozen at −80° C. until assaying using an ELISA as previously described (Osorio et al. 2003).

Results: Hepatitis B vaccine exposed to −20° C. for 18 hours became solidly frozen. Agglomeration of aluminum adjuvant and accelerated sedimentation of the vaccine in the vials were confirmed. Freeze-thaw of hepatitis B vaccine in saline without an excipient caused significant reduction in its immunogenicity (FIG. 3). The geometric mean antibody titer in mice receiving the frozen vaccine (without a protection excipient) is approximately 5-fold lower than the control vaccine stored at 4° C. Vaccines containing 50% glycerin, polyethylene glycol, or propylene glycol stored at 4° C. without freeze-thaw treatment elicited comparable antibody titers as the control vaccine, suggesting that these excipients had no adverse effect on the immunogenicity of the hepatitis B vaccine. All three excipients at 50% concentration prevented freezing of hepatitis B vaccine when exposed to −20° C. and prevented freeze-damage of the hepatitis B vaccine in the antibody readout. The antibody titers in mice immunized with vaccine containing 50% excipient that underwent three freeze-thaw treatments are comparable to that of the standard vaccine control and are significantly higher than that of frozen hepatitis B vaccine without an excipient (p<0.05, t test). This study indicated that glycerin, polyethylene glycol, and propylene glycol are compatible with hepatitis B vaccine and protect the vaccine against freeze-damage.

### Example #8

### Preservation of the in vitro Stability of Hepatitis B Vaccine After Freeze-Thaw

Method: The vaccine formulations containing glycerin, PEG-300, or propylene glycol were studied using standard potency assay to determine whether or not the excipients may compromise the stability of hepatitis B vaccine and its shelf life under normal storage condition. The vaccine preparation method is as described in Example 7. The concentration of excipients was 50% during freeze-thaw treatment and the final concentration was 10% during incubation at 4° C. Aliquots of formulations were analyzed on months 0, 1, 2, and 3 using AUZYME assays (Abbott Laboratories, Abbott Park, Ill.) by following the manufacturer's instruction. The AUZYME assay measures the in vitro potency of the vaccine by measuring the amount of hepatitis B surface antigen (HBsAg) of the vaccine.

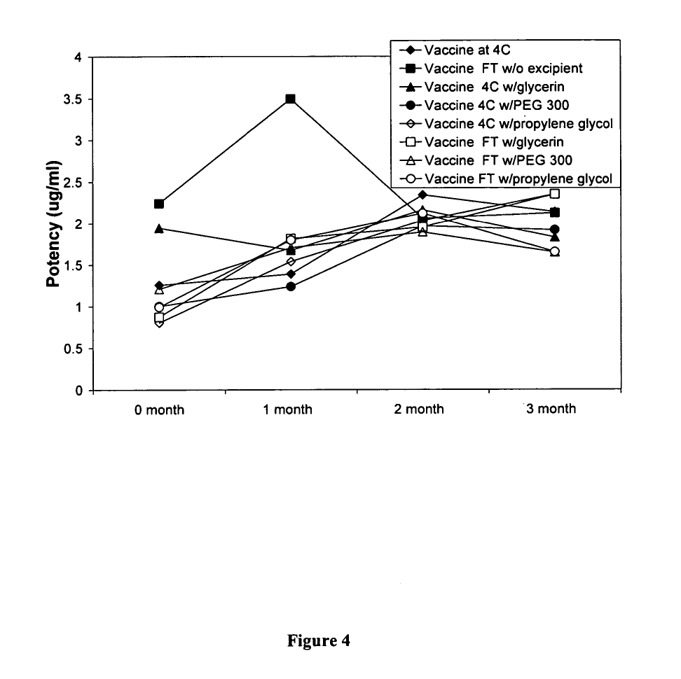

Results: The result of a three-month stability study is shown in FIG. 4 which depicts the effect of freeze-protection excipients on the stability of hepatitis B vaccine.

The data are the measured potencies with a target potency of 2 μg/ml.

Freeze-thaw of hepatitis B vaccine without an excipient did not change the in-vitro potency (amount of HBsAg). Further, the stability of HBsAg during the 3-month stability study at 4° C. is not affected by the initial freeze-thaw event. All three excipients, while protecting the aluminum adjuvant against freezing damage, have no adverse effect on the in-vitro potency and the stability of the hepatitis vaccine during the 3-month storage at 4° C. It is concluded that glycerin, PEG-300, and propylene glycol are compatible with HBsAg and hepatitis B vaccine by providing freeze protection without adversely affecting the stability of the protein antigen during freeze-thaw or storage at refrigeration temperature.

### Example #9

### Enhanced Stability of Hepatitis B Vaccine After Freeze-Thaw Cycles Followed by Exposure at Elevated Temperatures

Method: To determine if the freeze-protection excipients may confer heat stability to the hepatitis B vaccine, vaccines containing 50%, 30%, and 10% propylene glycol, were subjected to three freeze-thaw treatments then incubated at 45° C. for 21 days. The potencies of the vaccine formulations immediately after the freeze thaw cycles and at days 7 and 21 were determined using AUZYME assay and normalized to a standard vaccine stored at 4° C., which did not receive freeze-thaw treatment or heat exposure. A vaccine without an excipient that underwent freeze-thaw and incubated at 45° C. for 21 days was included as control.

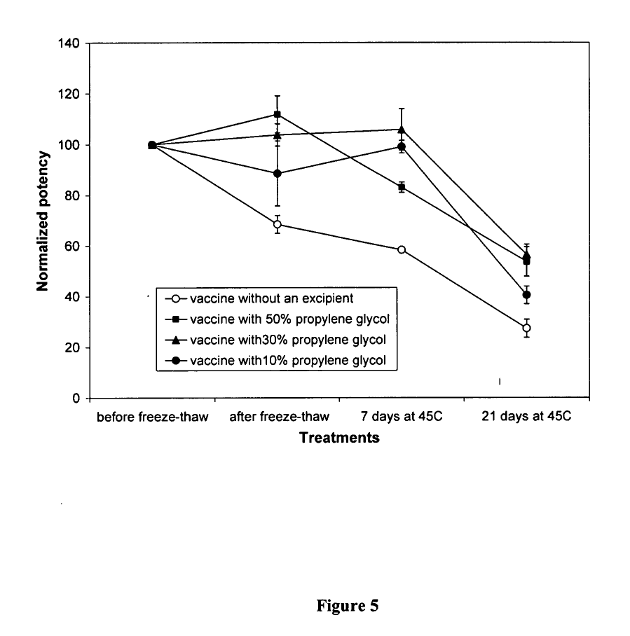

Results: During the freeze-thaw treatments, the vaccine without an excipient and vaccines with 30% and 10% propylene glycol were frozen and the 50% propylene glycol formulation did not freeze. The normalized potency data is shown in FIG. 5. Data are the average (n=3) potencies at each time point determined by AUZYME assay. There was about 30% potency loss with hepatitis B vaccine when exposed to three freeze-thaw treatments in the absence of a protection excipient. Immediately after freeze-thaw, all vaccine formulations containing propylene glycol have no potency loss. Further, vaccines containing propylene glycol at any tested concentration had normal potency at day 7 while the potency of the no excipient control experienced further loss. After 21 days of incubation at 45° C., all vaccine formulations experienced significant potency reduction. However, vaccines with propylene glycol had significantly higher remaining potency than the no excipient control. This result indicates that propylene glycol not only protects the vaccine against freeze-damage, but may also enhance the stability of the vaccine at elevated temperatures.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

What is claimed is:

1. A method of preventing damage to an adjuvant in a temperature sensitive adjuvanted liquid vaccine composition comprising:
adding a temperature protective agent to a first liquid vaccine composition to form a second liquid vaccine composition having a freezing point below the freezing point of said first liquid vaccine composition;
wherein said first liquid vaccine composition comprises an antigen and an adjuvant.

2. The method of claim 1, wherein the addition of said temperature protective agent lowers the freezing point of said first liquid vaccine composition below about 0° C.

3. The method of claim 2, wherein the addition of said temperature protective agent lowers the freezing point of said first liquid vaccine composition to about 0° C. to about −55° C.

4. The method of claim 1, wherein after said adding step, said temperature protective agent comprises about 1% to about 80% by volume of said second liquid vaccine composition.

5. The method of claim 1, further comprising storing said second liquid vaccine composition at about 0° C. to about −55° C. to protect said second liquid vaccine composition from microbial growth.

6. The method of claim 1, wherein said temperature protective agent comprises glycerin.

7. The method of claim 1, wherein said temperature protective agent comprises polyethylene glycol.

8. The method of claim 7, wherein said polyethylene glycol has an average molecular weight of about 300.

9. The method of claim 1, wherein said temperature protective agent comprises propylene glycol.

10. The method of claim 1, wherein said adjuvant is an aluminum salt.

11. The method of claim 10, wherein said adjuvant is aluminum hydroxide.

12. The method of claim 10, wherein said adjuvant is aluminum phosphate.

13. The method of claim 10, wherein said adjuvant is aluminum potassium sulfate.

14. The method of claim 1, wherein said adjuvant is calcium phosphate.

15. The method of claim 1, wherein said liquid vaccine composition is a human vaccine composition.

16. The method of claim 1, wherein said liquid vaccine composition is an animal vaccine composition.

17. A method of stabilizing a temperature sensitive adjuvanted liquid vaccine composition when frozen comprising:
adding a temperature protective agent to a first liquid vaccine composition to form a second liquid vaccine composition prior to freezing said first liquid vaccine composition;
wherein said first liquid vaccine composition comprises an antigen and an adjuvant; and
wherein said temperature protective agent is selected from the group consisting of glycerin, propylene glycol and polyethylene glycol.

18. The method of claim 17, wherein said temperature protective agent is polyethylene glycol having an average molecular weight of about 300 kD.

19. The method of claim 17, wherein said adjuvant is stabilized against agglomeration.

20. The method of claim 17, wherein said adjuvant is stabilized against sedimentation.

21. A method of stabilizing a temperature sensitive adjuvanted liquid vaccine composition at high temperatures comprising:
adding a temperature protective agent to a first liquid vaccine composition to form a second liquid vaccine composition prior to exposing said first liquid vaccine composition to high temperatures;
wherein said first liquid vaccine composition comprises an antigen and an adjuvant.

22. The method of claim 21, wherein said temperature protective agent protects said second liquid vaccine composition from temperature damage at temperatures up to about 55 ° C.

23. The method of claim 22, wherein said temperature protective agent protects said second liquid vaccine composition from temperature damage at temperatures from about 4° C. to about 55° C.

24. The method of claim 21, wherein said temperature protective agent is propylene glycol.
